(19) United States
(12) Patent Application Publication
(10) Pub. No.: US 2025/0135079 A1
(43) Pub. Date: May 1, 2025

(54) Method for Treating Sepsis

(71) Applicant: ELIAZ THERAPEUTICS, INC., Sebastopol, CA (US)

(72) Inventor: Isaac ELIAZ, Sebastopol, CA (US)

(21) Appl. No.: 18/827,781

(22) Filed: Sep. 8, 2024

Related U.S. Application Data
(60) Provisional application No. 63/546,447, filed on Oct. 30, 2023.

(51) Int. Cl. A61M 1/34

(52) U.S. Cl. CPC A61M 1/3496; A61M 1/3486

(57) ABSTRACT

A method for treating sepsis in a mammalian patient in need of such treatment is disclosed which relies on apheresis to achieve selective withdrawal of Galectin-3 to control or lower levels of Gal-3, which is shown to be a driver of the inflammatory cascade cycle in humans. Data and findings are based on a trial in humans, a porcine trial and a rat trial. Acute kidney injury, often associate in mammals with sepsis, is also shown to be effectively treated through the disclosed and claimed method. Treatment of AKI, independent of sepsis, through apheresis to achieve selective withdrawal of Gal-3 is also shown to be effective.

### PRIORITY DATA AND INCORPORATION BY REFERENCE

This application claims benefit of priority to U.S. Provisional Patent Application No. 63/546,447 filed Oct. 30, 2023. The entire disclosure of that application is incorporated herein by reference, without limitation.

### BACKGROUND OF THE INVENTION

### Field of the Invention

This invention pertains to treatment of sepsis. Sepsis is a life-threatening condition caused by a dysregulated inflammatory response to infection. Despite accounting for nearly twenty percent (20%) of all deaths worldwide, no safe and efficacious targeted therapies for sepsis exist within the clinical setting. Here, we disclose a method for treating mammals for sepsis. The treatment described and disclosed herein, while based on a three-part study evaluating the association between Galectin-3 (Gal-3) and sepsis, sepsis-associated mortality, and acute kidney injury (AKI) is in fact a complete treatment. The validation and study is described hereinbelow.

As noted, the study and validation relied upon herein is based on three parts. First, in a human cohort of patients admitted to an intensive care unit (ICU) with sepsis, mean serum Gal-3 concentration was significantly higher than in healthy controls. In the sepsis patients' group, Gal-3 concentration significantly increased over time in non-survivors compared to survivors. Gal-3 increase was associated with AKI as well.

Second, in a rat model of sepsis, Gal-3 removal by apheresis significantly improved survival. Treatment details are set forth hereinbelow, but survival was markedly increased, marking the first treatment so characterizing a mammalian patient.

Third, in a porcine model of sepsis, survival also significantly improved, and sepsis severity was significantly lower in pigs that underwent Gal-3 removal, as evidenced by significantly lower Extravascular Lung Water Index (ELWI), lactate levels, as well as significantly lower use of pressors and IV fluids, compared to controls. Furthermore, Gal-3 removal resulted in reduced tissue pathology in lung, heart, kidney, and liver tissue. Our findings demonstrate that Gal-3 removal presents a targeted therapy in the management of Sepsis, sepsis associated organ damage (multiple organ dysfunction syndrome (MODS), and Sepsis associated AKI (SA-AKI) as disclosed herein.

### Background of the Invention

Sepsis is a life-threatening condition caused by a dysregulated inflammatory response to infection, and is associated with a significant global burden of disease, causing approximately eleven (11) million deaths per year, representing approximately twenty percent (20%) of all deaths worldwide. Sepsis can lead to septic shock and multiple organ dysfunction syndrome (MODS), including acute respiratory distress syndrome (ARDS), acute kidney injury (AKI), and hepatic dysfunction. Sepsis-associated AKI is a common complication of sepsis that accounts for fifty percent (50%) of AKI cases in intensive care units (ICUs) and is associated with higher risk of long-term kidney disease and mortality compared to other types of AKI.

Currently, sepsis management primarily entails treating infections with intravenous antibiotics, volume repletion with intravenous fluids, and additional organ support (12-14). Given the high incidence of sepsis-related morbidity and mortality, novel therapies targeting the underlying pathophysiology of sepsis are imperative. Given the central role of the inflammatory response in sepsis, immune system modulation is a key target for potential therapies. While several therapies have been investigated, including statins, activated C protein, and monoclonal antibodies, there are no safe and efficacious targeted therapies for sepsis approved for use in a clinical setting.

Gal-3 is a glycan-binding protein of the lectin family secreted by monocytes, macrophages, and epithelial cells in response to cell and tissue damage and inflammation. Gal-3 is involved in the regulation of inflammation, immune responses, tissue repair, and cell death and affects the activation and release of pro-inflammatory cytokines, including interleukin (IL)-1, IL-6, nuclear factor kappa-light-chain-enhancer of activated B cells (NFκB) and tumor necrosis factor-α (TNF-α).

Substantial evidence has accumulated to suggest that Gal-3 is a key driving factor in cytokine storms during sepsis and contributes to immune dysregulation through endothelial cell activation, leading to vascular leakage and organ dysfunction. Among patients with sepsis, elevated serum Gal-3 concentrations are associated with poorer outcomes, including higher mortality rates. Additionally, Gal-3 directly induces renal tubular cell apoptosis, causing kidney inflammation and injury, and is a significant independent predictor of SA-AKI. Lower Gal-3 activity in animal models is associated with reduced sepsis rates and improved sepsis-related outcomes, as well as reduced AKI severity and rate. Furthermore, inhibition of Gal-3 is associated with significantly lower mortality and AKI incidence in rat models of sepsis compared to controls. Elevated serum Gal-3 concentrations have been shown to precede increases in serum IL-6 and creatinine, highlighting Gal-3 as an early-stage biomarker for sepsis-associated AKI and a driver of the inflammatory cascade.

Plasma adsorption utilizes adsorption columns to selectively withdraw and thereby remove potentially harmful molecules from plasma, such as cytokines and other inflammatory mediators. Plasma adsorption has previously been evaluated in sepsis models and inflammatory conditions. We previously developed an apheresis method using selective withdrawal of Gal-3 for plasma adsorption designed to deplete Gal-3, which effectively reduced serum Gal-3 concentrations. See, for example, U.S. Pat. No. 10,953,148.

Herein we describe the relations among serum Gal-3 concentrations, sepsis-related organ injury, and mortality in a cohort of patients with sepsis and demonstrate the effects of a Gal-3 adsorption column in two animal models of sepsis. Our research has demonstrated that the same method, selective withdrawal of Gal-3 through apheresis, is an effective treatment of sepsis-associated Acute Kidney Injury (AKI) and AKI independent of sepsis.

This invention provides a treatment for sepsis, a devastating disease often encountered in the very sites where other diseases are treated—hospitals, clinics and the like. Wherever it is deployed, this invention provides for, as an initial step, that a mammalian patient, typically a human, first be identified as one experiencing the disease sepsis. Sepsis, as those of skill in the art are aware, is diagnosed best through a wholistic approach.

To effectively identify mammalian patients in need of treatment for sepsis, a definition that incorporates both clinical criteria and diagnostic markers is most effectively employed. These are set forth in more detail immediately below.

### A) Clinical Signs and Symptoms:

Fever (hyperthermia) or hypothermia.

Tachycardia or bradycardia, depending on age and baseline heart rate.

Tachypnea or altered respiratory rate appropriate to age.

Altered mental status ranging from confusion to coma.

Hypotension or signs of peripheral shutdown (e.g., cold extremities, prolonged capillary refill time) indicative of impending or existing shock.

### B) Laboratory Indicators:

Elevated markers of inflammation such as CRP or procalcitonin.

Evidence of organ dysfunction, such as elevated serum creatinine, abnormal liver function tests, thrombocytopenia, or coagulation.

Lactic acidosis or elevated serum lactate, indicating tissue hypoperfusion.

### C) Evidence of Infection:

Positive culture or other evidence of infection from blood or other sterile sites.

Clinical evidence suggesting a high probability of infection (e.g., purulent discharge, imaging findings).

### D) Risk Factors:

Patients with existing chronic conditions such as diabetes, immunosuppression, cancer, chronic organ dysfunction, or recent surgery.

Neonates and the elderly due to their vulnerable immune status.

Patients with recent exposure to invasive procedures, indwelling devices, or hospitalization, which increase the risk of healthcare-associated infections.

### E) Immediate Treatment Requirement:

The need for rapid antimicrobial therapy initiation within the first hours of recognition. Fluid resuscitation and hemodynamic support as indicated by clinical assessment. This is accompanied by monitoring and support of organ function in an intensive care setting if and while needed.

No one factor is determinative in identifying septic patients, but sepsis is neither newly discovered nor undocumented. This invention begins with identification of a mammalian patient, which may be a human, in need of treatment for sepsis. Those of skill in the art are well acquainted with the indications of, and the identification of, sepsis. What has been unavailable previously is an effective treatment for sepsis.

### SUMMARY OF THE INVENTION

Applicant has determined that effective treatment of sepsis in mammals, including humans, can be achieved by selective withdrawal of Gal-3 through apheresis. This ex vivo treatment of blood, to lower the concentration of Galectin-3 (GAL-3) has been demonstrated, in established animal models, and with treatment of a human cohort, to effectively treat and cure sepsis. Given that sepsis is frequently contracted and communicated in a hospital setting, apheresis treatment does not present issues in bringing the patient to a treatment center.

To effectively treat sepsis, a patient is treated with apheresis. There are a wide number of apheresis protocols and treatments known, for various purposes. To effectively treat patients to overcome sepsis, the apheresis may be of blood (plasma) separated into separate portions, so as to remove one or more of white blood cells, platelets, red blood cells and plasma. Details and protocols for apheresis vary widely, but are neatly summarized and detailed in U.S. Pat. No. 10,953,148, assigned to Eliaz Therapeutics, Inc. of California. Recently, the same researchers have made strides in treatment using whole blood apheresis, as set forth in detail in U.S. patent application Ser. No. 17/964,644. Thus, it is also possible now to treat certain cases with whole blood apheresis, depending on targets and conditions. The disclosure of both U.S. Pat. No. 10,953,148 and U.S. patent application Ser. No. 17/964,644 are both incorporated herein-by-reference in their entirety.

Apheresis is a technique developed to treat a variety of conditions and pathologies. Many of these treatments are effected by removing one type of blood, one agent or one component of the blood, and returning the remainder to the patient. Whether the apheresis is of whole blood or the more conventional treatment following separation the target in this invention for the treatment of sepsis is Galectin-3, referred to herein as Gal-3.

Gal-3 has been shown to be an upstream mediator of the inflammatory cascade underlying sepsis. By intervening to lower levels of Gal-3 in the blood by at least about 12%, treatment of septic patients has been shown to be effective in reversing their condition, as demonstrated in this application. This process of using apheresis to remove component of blood, in this case gal-3, has come to be referred to as “selective withdrawal” of the target.

Average Gal-3 blood levels in healthy adults typically range from 8 to 15 ng/ml. In patients with sepsis, Gal-3 levels are usually significantly elevated beyond this range. The effectiveness of apheresis in reducing Gal-3 levels in sepsis patients can vary; while selective withdrawal of Galectin-3 through apheresis will result in a notable decrease across the column, the levels of Gal-3 in the blood may not reflect it in the initial & acute phase, as patients will likely continue to produce excess Gal-3 during the initial period of their illness. Generally, Gal-3 apheresis treatments in sepsis should be administered initially for 1 to 3 consecutive days, with a preference for 1 to 5 days, depending on the patient's Gal-3 levels and clinical outcome following initial treatment. Additional treatments may be considered based on the patient's Gal-3 levels and clinical response after the initial treatment series. The invention remarkably has demonstrated effective results for some patients following a single day of treatment.

A principal focus of this invention is the successful treatment of sepsis, by reducing sepsis-related gal-3 levels in a patient's blood. As discussed throughout this application, Acute Kidney Injury, or AKI, is closely associated with, and coincident with sepsis. The effect and events associated with AKI due to sepsis are also effectively treated by apheresis to reduce gal-3 levels. Thus, while the invention disclosed herein is sometimes referred to as an effective treatment for sepsis, and in other passages as effective in addressing damage due to AKI, it is an effective treatment—relying on the reduction of gal-3 levels through selective withdrawal of gal-3 through apheresis, which is also an effective treatment for sepsis associated AKI. Selective withdrawal is effected by passing the patient's blood in the apheresis device over am array of binding agents secured to the apheresis device interior. The binding agents are typically anti-bodies, ether natural or recombinant, that effectively bind to galectin-3. The Gal-3 so bound stays in the apheresis tube while the blood is passed through and thereafter returned to the patient. The binding agent may be a natural protein and the like if the binding coefficient is high enough.

The effectiveness of this invention is demonstrated repeatedly. Applicant has demonstrated this invention using a rat model, in porcine trials and finally in a human cohort trial. Human trials are always of the greatest impact and call out for the greatest focus—the human trials are set forth immediately below. Thereafter, trials using a well-established rat model of sepsis are discussed in detail. Finally, a detailed study of a porcine model of sepsis is set forth. The combination of human, rat and porcine trials provides a compelling showing of the effectiveness and value of the claimed invention.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 illustrates measured serum levels of IL-6, Gal-3 and Creatinine in patients with sepsis as well as controls.

FIG. 2 illustrates serum concentrations of IL-6, Gal-3 and Creatinine (Cr) in sepsis survivors compared with levels in non-survivors.

FIG. 3 illustrates serum concentrations of IL-6, Gal-3, and Cr on days 1, 2, and 3 in sepsis patients with AKI (n=33) and without AKI (non-AKI, n=54).

FIG. 4 illustrates percent survival in a rat CLP model of sepsis with and without Gal-3 whole blood apheresis (n=10 and 9, respectively).

FIG. 5 illustrates values for the Extravascular Lung Water Index (ELWI) over time in a porcine lipopolysaccharide injection (LPS) model of sepsis with Gal-3 apheresis compared to controls.

FIG. 6 illustrates lactate concentrations in a porcine model of sepsis with Gal-3 apheresis (treatment group) compared to controls.

FIG. 7 illustrates serum Gal-3 concentration in the treatment and sham (control) groups at baseline, post-LPS, and the following 1, 2 and 3 hours of treatment.

FIG. 8 illustrates serum IL-6 concentration (pg/ml) in the treatment and sham control groups at baseline, 0-hour, 1 hour, 2 hours and 3 hours post-LPS.

FIG. 9 illustrates percent survival in a porcine LPS model of sepsis with and without Gal-3 apheresis.

FIGS. 10 A-L illustrate comparative histological samples across four different tissue types.

### DETAILED DESCRIPTION OF THE INVENTION

As noted above, this invention is disclosed in terms of the results obtained through three different and inter-supporting trials. Applicant's invention is told through the details of a substantial human cohort trial. Since human trials always command the most specific and exact attention, the human trials are reported below. Following the human cohort trials, we report on trials involving a well-established rat model. The third trial relied upon and reported herein is a porcine sepsis model trial. Following the report of the porcine model trial, there is further discussive detail of the invention provided.

### Part 1: Human Cohort

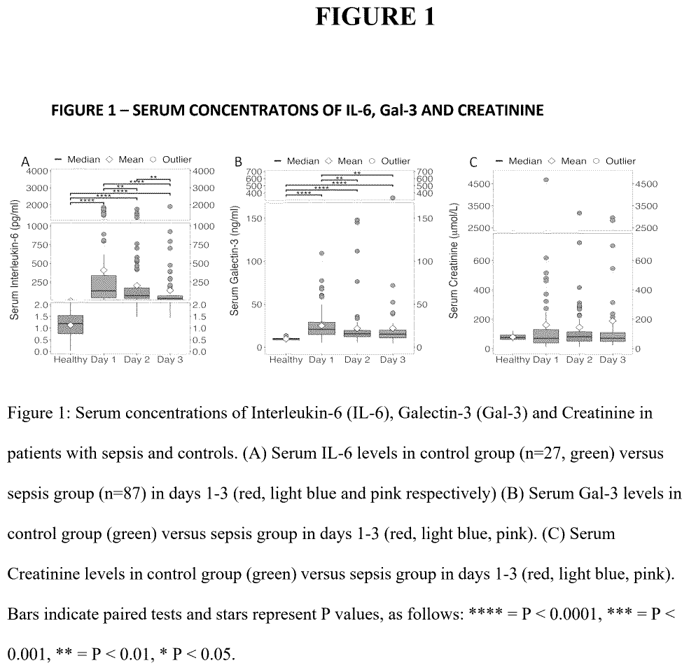

In total, one hundred fourteen (114) participants were included in the study, including eighty-seven (87) patients with sepsis and twenty-seven (27) healthy controls. The sepsis group exhibited significantly elevated serum concentrations of Gal-3 and IL-6 at all time points compared to the control group and remained significant after excluding patients with diabetes (p<0.001) (Table 1; FIGS. 1A and 1C). Patients with sepsis demonstrated mean serum Gal-3 concentrations of 20.68 ng/ml, 15.49 ng/ml, and 15.13 ng/ml on Day 1 (day of admission to the ICU), Day 2, and Day 3 respectively compared to a mean concentration of 9.66 ng/ml in the control group (P<0.001) (Table 1; FIGS. 1A and 1B). There were no significant differences in serum creatinine concentrations between the sepsis and control groups at all time points (Table 1; FIG. 1C; all P>0.05).

TABLE 1
Characteristics of patients in the control group and sepsis group.

| | Healthy Patients | | Sepsis Patients | | |
|---|---|---|---|---|---|
| Variables | (n = 27) | | (n = 87) | | p value |
| Age | 54.0 | (50.0-60.5) | 64.0 | (53.5-74.0) | 0.004 |
| Proportion | 0.37 | (0.28-0.47) | 0.41 | (0.36-0.47) | 0.880 |
| Female | | | | | |
| Serum Cr. | 74.27 | (62.5-92.47) | 69.05 | (38.13-129.61) | 0.295 |
| Day 1 | | | | | |
| Serum Cr. | | | 80.15 | (50.03-114.89) | 0.856 |
| Day 2 | | | | | |
| Serum Cr. | | | 69.79 | (48.89-110.01) | 0.680 |
| Day 3 | | | | | |
| Serum Gal-3 | 9.66 | (8.4-10.39) | 20.68 | (14.61-29.24) | <0.001 |
| Day 1 | | | | | |
| Serum Gal-3 | | | 15.49 | (12.17-19.63) | <0.001 |
| Day 2 | | | | | |
| Serum Gal-3 | | | 15.13 | (10.88-19.87) | <0.001 |
| Day 3 | | | | | |
| IL6 Day 1 | 1.18 | (0.76-1.54) | 130.94 | (39.84-337.81) | <0.001 |
| IL6 Day 2 | | | 66.04 | (30.93-173.11) | <0.001 |
| IL6 Day 3 | | | 29.85 | (14.28-66.38) | <0.001 |

Note:
Data are shown as median values, with interquartile ranges spanning from the 25th to the 75th percentile.

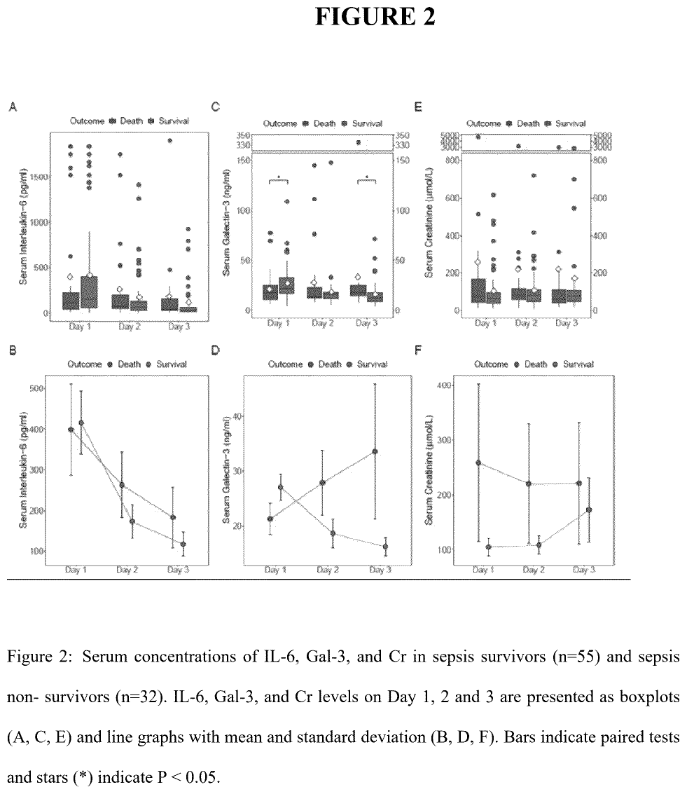

In survivors, the mean serum Gal-3 concentration decreased on Day 2 and Day 3, while the mean serum Gal-3 concentration increased on Day 3 in non-survivors (FIG. 2). On Day 3, the mean serum Gal-3 concentration was significantly higher in the non-survivors group compared to survivors (P=0.007) (Table 1; FIG. 2B). Urine analysis for Gal-3 demonstrated no significant difference in urine Gal-3 excretion between survivors and non-survivors, as well as no significant change in Gal-3 excretion over time in each group.

The incidence of (all cause) AKI was similar among survivors and non-survivors (44% versus 35%) (Table 2). Moreover, there were no significant differences in mean serum creatinine, IL-6, and lactate concentration, SOFA score, or APACHE II score between survivors and non-survivors. (Table 2) (FIGS. 2B and 2F).

TABLE 2
Characteristics of sepsis patients who survived and did not survive.

| Variable | Whole Group (n = 87) | | Survivors (n = 55) | | Non-Survivors (n = 32) | | p value |
|---|---|---|---|---|---|---|---|
| Age | 64 | (53.5-74) | 64 | (53.5-72) | 69 | (56.5-76.25) | 0.16 |
| Proportion | 0.41 | (0.36-0.47) | 0.42 | (0.35-0.49) | 0.41 | (0.32-0.49) | 1 |
| Female | | | | | | | |
| Serum Cr. | 69.05 | (38.13-129.61) | 62.30 | (37.98-96.11) | 75.51 | (44.42-168.25) | 0.18 |
| Day 1 | | | | | | | |
| Serum Cr. | 80.15 | (50.03-114.89) | 78.99 | (48-113.73) | 80.15 | (54.67-117.21) | 0.72 |
| Day 2 | | | | | | | |
| Serum Cr. | 69.79 | (48.89-110.01) | 76.06 | (49.94-109.49) | 59.34 | (42.62-111.58) | 0.48 |
| Day 3 | | | | | | | |
| Serum | 20.68 | (14.61-29.24) | 21.82 | (16.89-33.19) | 17.52 | (10.63-25.24) | 0.03 |
| Gal-3 | | | | | | | |
| Day 1 | | | | | | | |
| Serum Gal- | 15.49 | (12.17-19.63) | 16.18 | (11.82-18.56) | 14.02 | (12.55-22.76) | 0.52 |
| 3 Day 2 | | | | | | | |
| Serum | 15.13 | (10.88-19.87) | 12.44 | (9.38-17.64) | 18.05 | (14.57-25.12) | 0.007 |
| Gal-3 | | | | | | | |
| Day 3 | | | | | | | |
| IL6 Day 1 | 130.94 | (39.84-337.81) | 149.57 | (54.87-407.44) | 111.8 | (39.67-221.45) | 0.52 |
| IL6 Day 2 | 66.04 | (30.93-173.11) | 59.44 | (25.98-127.53) | 75.26 | (44.81-200.77) | 0.25 |
| IL6 Day 3 | 29.85 | (14.28-66.38) | 27.12 | (13.03-64.28) | 37.58 | (22.98-157.58) | 0.37 |
| LAC | 2.3 | (1.7-3.9) | 2.2 | (1.4-3.4) | 2.8 | (1.8-4.4) | 0.164 |
| SOFA | 8.0 | (5.2-13.0) | 8.0 | (5.5-13.0) | 8.0 | (5.0-13.0) | 0.85 |
| APACHEII | 23.0 | (14.5-30.0) | 23.0 | (14.0-27.5) | 26.0 | (16.5-32.3) | 0.18 |
| AKI | 0.38 | (0.32-0.43) | 0.35 | (0.28-0.41) | 0.44 | (0.35-0.53) | 0.53 |
| Proportion | | | | | | | |

Note:
Data are shown as median values, with interquartile ranges spanning from the 25th to the 75th percentile

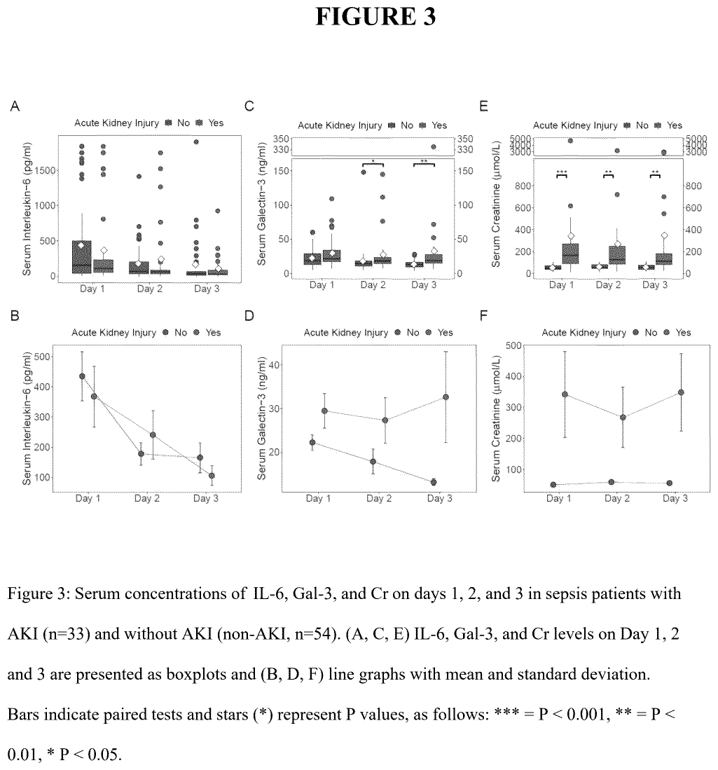

Patients with sepsis and sepsis-associated-AKI had significantly higher APACHE II scores, SOFA scores, and serum lactate compared to those without sepsis-associated-AKI (P<0.01; Table 3). Patients with sepsis-associated AKI exhibited significantly higher serum Gal-3 concentrations than those without sepsis associated AKI on days 2 (P=0.01) and 3 (P=0.005); (Table 3; FIGS. 3C and 3D). There were no significant differences in serum IL-6 concentrations among patients with and without sepsis-associated AKI at any time point, although levels decreased over time (Table 3; FIGS. 3A and 3B).

TABLE 3
Characteristics of sepsis patients with AKI and without AKI.

| Variable | Whole Group (n = 87) | | AKI (n = 33) | | Non-AKI (n = 54) | | p value |
|---|---|---|---|---|---|---|---|
| Age | 64.0 | (53.5-74.0) | 64 | (54-74) | 65 | (52.25-75.5) | 0.89 |
| Proportion Female | 0.41 | (0.36-0.47) | 0.36 | (0.28-0.45) | 0.44 | (0.38-0.51) | 0.60 |
| Serum Cr. Day 1 | 69.05 | (38.13-129.61) | 164.39 | (89.67-272.61) | 45.85 | (35.56-70.98) | <0.001 |
| Serum Cr. Day 2 | 80.15 | (50.03-114.89) | 124.16 | (83.62-248.09) | 53.7 | (41.93-80.73) | <0.001 |
| Serum Cr. Day 3 | 69.79 | (48.89-110.01) | 111.58 | (78.14-182.62) | 50.98 | (35.31-72.93) | <0.001 |
| Serum Gal-3 Day 1 | 20.68 | (14.61-29.24) | 21.33 | (17.19-34.39) | 18.35 | (12.88-29.07) | 0.21 |
| Serum Gal-3 Day | 2 15.5 | (12.17-19.63) | 18.10 | (14.20-23.45) | 13.98 | (10.26-18.50) | 0.01 |
| Serum Gal-3 Day 3 | 15.13 | (10.88-19.87) | 18.22 | (14.91-27.47) | 12.44 | (8.91-15.93) | 0.005 |
| IL6 Day 1 | 130.94 | (39.84-337.81) | 109.55 | (51.62-233.27) | 152.87 | (38.47-498.99) | 0.56 |
| IL6 Day 2 | 66.04 | (30.93-173.11) | 62.41 | (30.94-88.75) | 68.81 | (34.04-204.23) | 0.67 |
| IL6 Day 3 | 29.85 | (14.28-66.38) | 25.83 | (15.73-87.63) | 32.51 | (14.38-64.28) | 0.84 |
| LAC | 2.3 | (1.7-3.9) | 2.8 | (2.0-5.0) | 2.05 | (1.4-3.2) | 0.01 |
| SOFA | 8.0 | (5.2-13) | 13.0 | (8.0-15.0) | 7.0 | (4.0-10.0) | <0.001 |
| APACHEII | 23.0 | (14.5-30.0) | 27.0 | (21.0-33.0) | 18.0 | (13.0-26.0) | 0.002 |

Note:
Data are shown as median values, with interquartile ranges spanning from the 25th to the 75th percentile. Cr = Creatinine, Gal-3 = Galectin 3, IL6 = Interleukin 6, LAC = lactate, SOFA = sequential organ failure assessment, APACHE II = acute physiology and chronic health evaluation II.

### Trial 2: Rat CLP Model of Sepsis and Gal-3 Apheresis

Nineteen rats were subjected to CLP (cecal ligation puncture). One hour following the CLP procedure, ten animals underwent a 2-hour whole blood apheresis to remove Gal-3 while nine animals underwent a 2-hour sham whole blood apheresis.

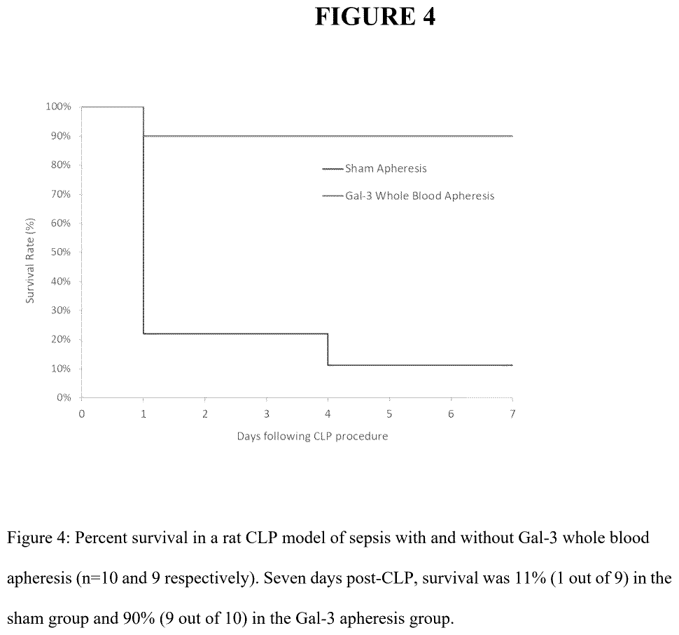

Nine out of the ten animals in the apheresis treatment group survived until the end of the seven-day study, while eight of the nine animals in the control group died by day 4 (P<0.001; FIG. 4).

### Part 3: Porcine LPS Model of Sepsis and Gal-3 Apheresis

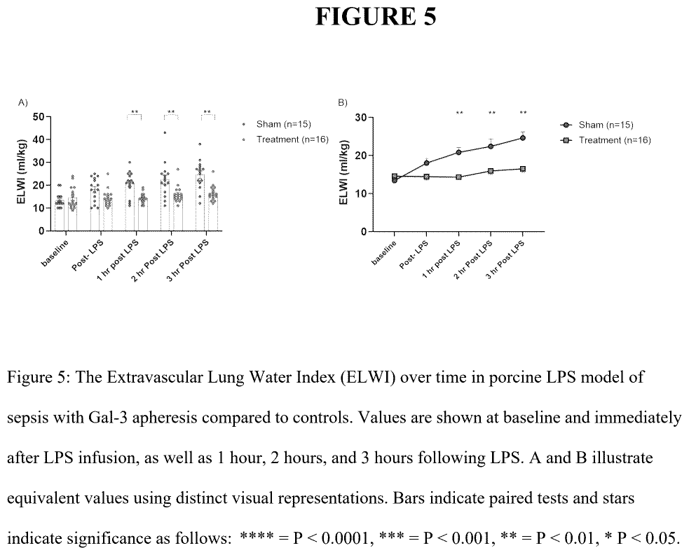

Following the results in the rat model, we studied the effects of Gal-3 apheresis with selective withdrawal of Gal-3 in a porcine LPS model of sepsis that more closely models human sepsis (41). Prior to LPS-mediated induction of sepsis, there was no significant difference in ELWI between the treatment and control groups. Following LPS induction, pigs treated with Gal-3 selective withdrawal apheresis demonstrated minimal rise in ELWI over time from baseline throughout the three-hour experiment period. In comparison, ELWI significantly increased over time in the porcine sham apheresis group beginning after the LPS infusion (P=0.021) and continued to rise significantly at 1, 2, and 3 hrs following induction of sepsis (P<0.001) (FIG. 5). Beginning at 1 hr following LPS infusion, the Gal-3 selective withdrawal apheresis group demonstrated a significantly lower ELWI compared to the sham group and remained significantly lower at 2- and 3-hr post LPS (1 hr: P=0.002; 2 hr: P=0.007; 3 hr: P=0.002) (FIG. 5).

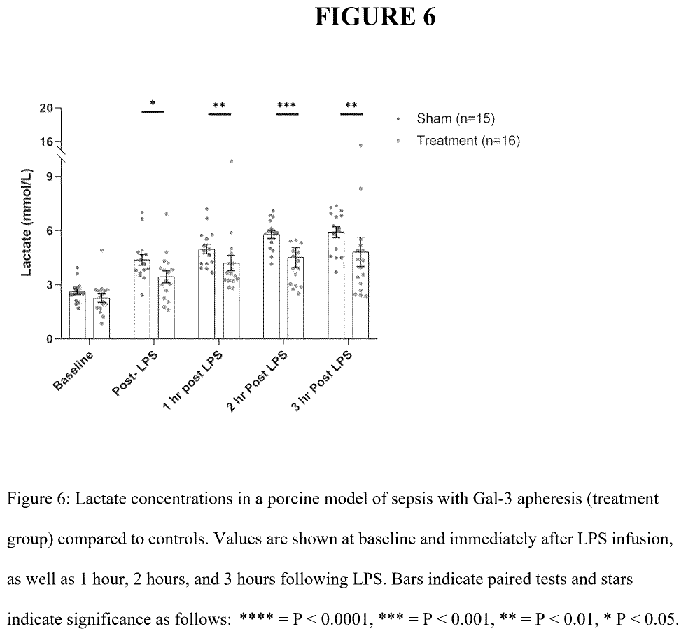

Prior to LPS infusion, there was no significant difference in serum lactate concentration between the porcine treatment and control groups. Following induction of sepsis, serum lactate concentration increased with sepsis induction in both groups and rose to a greater extent in the control group (FIG. 6). Beginning post LPS infusion, the mean serum lactate concentration was significantly lower in the Gal-3 apheresis group than in the sham group at all time points (P<0.05) (FIG. 6).

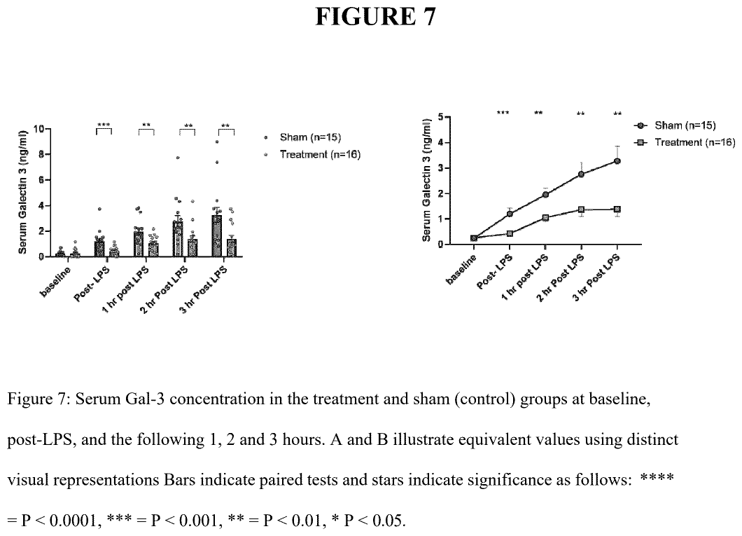

At baseline, there was no significant difference in mean serum Gal-3 concentrations between the Gal-3 selective withdrawal apheresis group and controls. Following induction of sepsis with LPS, the mean serum Gal-3 concentration was significantly lower in the Gal-3 apheresis group compared with controls (P=0.001) (FIG. 7). Serum Gal-3 concentrations remained significantly lower in the Gal-3 apheresis group compared to controls at 1 hr, 2 hr, and 3 hr following sepsis induction (1 hr: P=0.009; 2 hr: P=0.005; 3 hr: P=0.003) (FIG. 7).

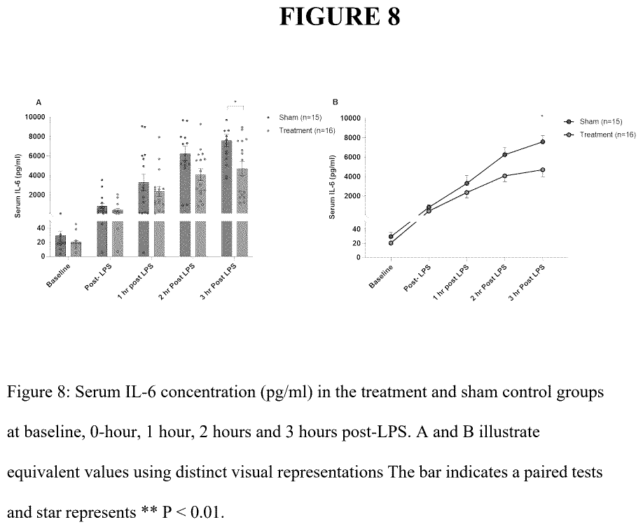

Prior to the LPS infusion, there was no significant difference in serum IL-6 concentration between the treatment group and the control group. After LPS-mediated induction of sepsis, the IL-6 concentration gradually rose in both groups with a significantly more rapid rise in the control group (FIG. 8). At 3 hr following sepsis induction, IL-6 was significantly lower in the treatment group compared to the control group (P=0.0076; FIG. 8). Additionally, both the total fluid requirement (P<0.001) and cumulative norepinephrine dose (P=0.0025) were significantly higher in the control group than the Gal-3 apheresis group.

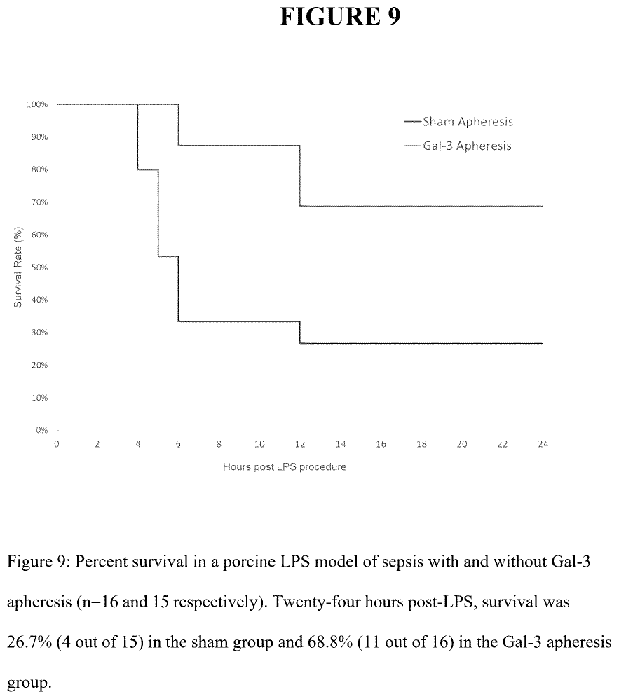

The porcine survival rate was significantly higher in the Gal-3 apheresis group (68.8%) compared to the control group (26.7%) over the 24-hr study period (P=0.004). The survival rate in the control group declined rapidly following LPS-mediated induction of sepsis, reaching 33.3% by 6-hr post LPS infusion and 26.7% at 12-hr post LPS (FIG. 9). In the treatment group, corresponding survival rates were 87.5% and 68.8% respectively (FIG. 9).

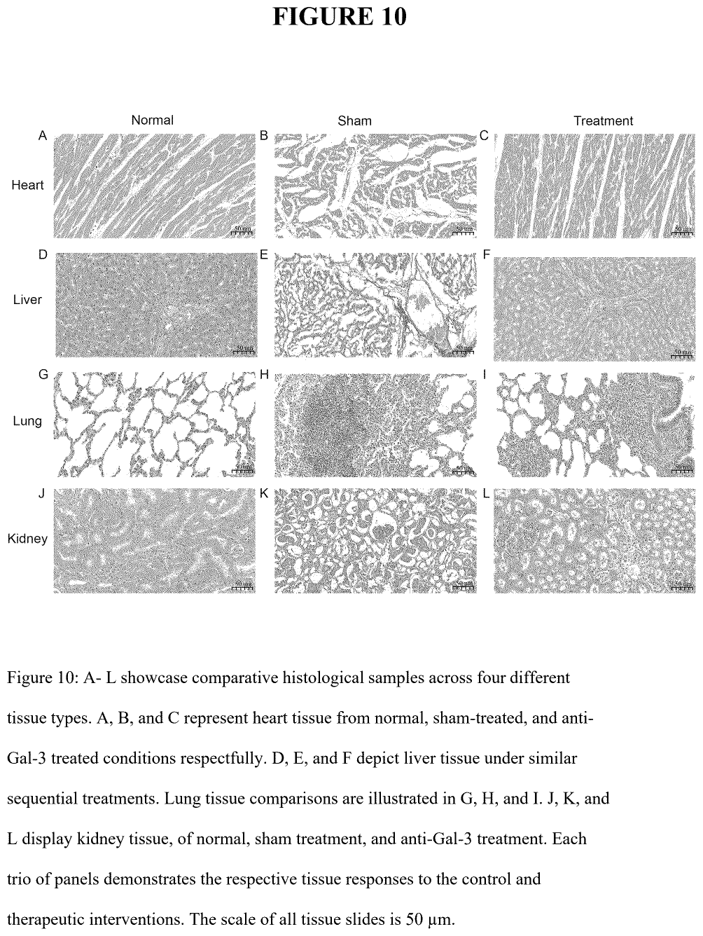

Histological analysis of tissue from the treatment group exhibited decreased pathological changes following LPS-mediated sepsis compared with controls (FIG. 10; Table 4). Compromised structural integrity, necrosis and immune cell infiltration were observed in all organ tissue samples of the control group (FIG. 10; Table 4), whereas necrosis and cell infiltration were absent from most organ tissue samples of the Gal-3 apheresis group (Table 4).

TABLE 4
Key observations from histological analysis of tissue
from porcine treatment group and control group.

| | Key observations | |
|---|---|---|
| Organ | Sham | Treatment |
| Heart | Disordered arrangement, widespread | Uniform cardiomyocyte morphology |
| (A-C) | shrinkage and deformation of | with clear cell boundaries. Normal |
| | cardiomyocytes. Irregular | interstitium. Absence of |
| | cardiomyocyte nuclear shapes. | inflammatory cell infiltration. |
| | Increased interstitial space. Occasional | |
| | granulocytes and lymphocytes | |
| | infiltration | |
| Liver | Widespread congestion and dilation of | Central vein located at the center of |
| (D-F) | hepatic sinusoids, disorganized hepatic | the hepatic lobule, hepatocytes and |
| | cord arrangement. Extensive shrinkage | hepatic sinusoids arranged in a |
| | and deformation of hepatocytes. | radiating pattern around the central |
| | Irregular shaping and pyknosis of | vein. Mild hepatocellular steatosis |
| | nuclei, small areas of hepatocellular | featuring small round cytoplasmic |
| | focal necrosis. Occasional | vacuoles. Widespread congestion |
| | lymphocytic infiltration around portal | and dilation of hepatic sinusoids |
| | triads | Absence of inflammatory cell |
| | | infiltration. |
| Lung | Compromised structural integrity, | Mild alveolar wall and septa |
| (G-I) | widespread necrosis, nuclear | thickening and widening, cell |
| | fragmentation, frequent alveolar | detachment and occasional necrosis, |
| | expansion, presence of macrophages, | small number macrophages and |
| | lymphocyte infiltration | granulocytes, white blood cells in |
| | | blood vessels, Lymphocyte |
| | | infiltration |
| Kidney | Widespread renal tubular epithelial | Minor necrosis and detachment of |
| (J-L) | cell necrosis and detachment. | renal tubular epithelial cells, |
| | Pyknosis of nuclei. Renal tubules | pyknosis of nuclei, uniform cell |
| | atrophy and renal cyst dilatation. | count and matrix in glomeruli. No |
| | Increased glomerulus and renal cyst | abnormalities in the interstitium of |
| | wall gap. Severe interstitial bleeding. | kidney tissue. Absence of |
| | Absence of inflammatory cell | inflammatory cell infiltration. |
| | infiltration. | |

### DISCUSSION

Herein we describe results of three distinct but complementary studies: 1) a cohort of patients with sepsis where we examined associations among clinical variables, Gal-3, and other inflammatory markers; 2) a cecal ligation and puncture model of sepsis in rats treated with a Gal-3 adsorption column (versus sham); and 3) an LPS model of sepsis in pigs treated with a Gal-3 adsorption column (versus sham). We found that a persistent increase in serum Gal-3 concentrations was associated with progression (or delayed resolution) of sepsis, sepsis-associated acute kidney injury, and sepsis-related mortality in a cohort of patients with sepsis. In animal models of sepsis, Gal-3 removal using selective withdrawal apheresis significantly decreased sepsis severity, decreased associated mortality, and reduced pathological changes on evaluation of tissue histology.

Results from our cohort study are consistent with prior studies exploring the role of Gal-3 in the pathogenesis of sepsis, sepsis-associated AKI, and sepsis-related mortality (21, 24, 30, 31, 33). Serum Gal-3 and IL-6 concentrations were elevated in patients admitted to the ICU with sepsis compared to controls. Among sepsis non-survivors, serum Gal-3 concentrations increased over time and were significantly higher than Gal-3 concentrations in survivors on Day 3. The findings demonstrate an association between elevated Gal-3 and sepsis, sepsis mortality, and AKI.

In a validated porcine model of sepsis induction using LPS, we found that Gal-3 levels were significantly reduced at all time points following apheresis compared with controls, demonstrating successful removal of Gal-3 (42). Gal-3 removal resulted in significantly lower sepsis severity compared with controls as evidenced by both ELWI and serum lactate concentrations. Notably, following LPS infusion and Gal-3 apheresis, there was no significant increase in ELWI in the Gal-3 apheresis group at any time point, while ELWI in the control group continued to deteriorate at each time point following LPS infusion. Similarly, Gal-3 removal attenuated the rise in serum lactate in the Gal-3 apheresis group compared with controls. Notably, the fluid and pressor requirements in the control group were also significantly greater than in the Gal-3 apheresis group. These findings demonstrate that Gal-3 removal in a porcine model using apheresis significantly reduces sepsis severity as evidenced by both clinical measures and biomarkers.

Recent studies have highlighted the role of Gal-3 in the inflammatory response (21, 24, 31, 33). Gal-3 regulates immune cells and pro-inflammatory cytokines, such as interleukin-6 (IL-6) and tumor necrosis factor-a (TNF-α), leads to internalization of endotoxins such as LPS, and directly induces renal tubular cell apoptosis (23, 25, 26, 31, 32). We found that Gal-3 removal resulted in a significant reduction in IL-6 at 3 hour following induction of sepsis compared to controls, consistent with previous findings in animal models treated with Gal-3 inhibitors (23, 33). The delayed trend in IL-6 reduction following Gal-3 removal, in combination with the rapid reduction in sepsis severity, illustrates the role of Gal-3 as an upstream mediator of the inflammatory cascade underlying sepsis (33).

In the present study, organ tissue histopathology in the Gal-3 apheresis group exhibited markedly reduced tissue necrosis and improved structural integrity compared with controls (43). Among controls, lung, heart, kidney, and liver tissue showed compromised structural integrity and necrosis with evidence of widespread necrosis in lung tissue and frequent alveolar expansion, disordered and deformed cardiomyocytes, widespread renal tubular epithelial cell necrosis with severe interstitial bleeding, and extensive deformation of hepatocytes, among other findings. In contrast, in the Gal-3 apheresis group, there was only occasional or mild necrosis in lung and kidney tissue, hepatic congestion with dilation of hepatic sinusoids, and normal cardiomyocyte morphology. The reduction in tissue pathology corresponds with the reduction in sepsis severity seen in the Gal-3 apheresis group. While the study period and post-mortem histopathology analysis limited follow-up evaluation in the porcine model, these findings suggest that Gal-3 removal not only ameliorates sepsis severity but may also reduce chronic morbidity and organ dysfunction following sepsis (30, 44).

In both rat and porcine models of sepsis, Gal-3 removal via adsorption columns for selective withdrawal of Gal-3 resulted in significantly improved survival compared to controls. Our findings confirm and extend previously published findings suggesting a role of Gal-3 in mortality and other complications of sepsis, by demonstrating that reduction in serum Gal-3 concentrations by column adsorption attenuates many of the adverse effects of sepsis on organ structure and function. Taken together with observational data from humans, Gal-3 removal via selective withdrawal using apheresis could represent a novel, extracorporeal approach toward managing sepsis and associated complications (including AKI) in critically ill patients.

Given the pathophysiology of sepsis, novel therapies targeting immune dysregulation require further exploration. As evident in prior studies and in our current findings, Gal-3 presents a unique target in the treatment of sepsis (15, 16, 21, 32, 34, 45). We demonstrated that Gal-3 can be effectively removed in a porcine model of sepsis using apheresis. Following the completion of the porcine study we were able to significantly improve the Gal-3 depletion efficiency and capacity of the XGAL-3 column. We expect this improvement to yield better clinical results. Gal-3 removal significantly reduced sepsis severity, organ damage, and mortality. Given these findings, Gal-3 removal using apheresis presents a potential future therapy in the treatment of sepsis. Future research is warranted to further explore the mechanism of Gal-3 in sepsis pathogenesis and refine approaches to Gal-3 removal as a potential directed therapy in the management of sepsis.

Typically, patents and patent applications do not faithfully reproduce all details and information regarding the methods employed to arrive at the inventions disclosed. Because this invention, the treatment of sepsis and sepsis associated AKI is premised on three very different and reinforcing studies and the method steps employed therein, we present here the methods employed in detail. The detailed method information allows one of skill in the art to compare and contrast the methods employed, and the results obtained, allowing independent confirmation of the results and implications.

### Human Cohort

### Patient Recruitment and Surviorship

We recruited patients (n=87) with a diagnosis of septic shock from the intensive care unit (ICU) of Zhongnan Hospital of Wuhan University, Hubei Province, China, between August 2020 and December 2022. We recruited healthy volunteers (n=27) from the general population of Hubei province, China. Our study examined patients that identified as male and female, and we analyzed the distribution among patients with different clinical outcomes. The diagnosis of sepsis was based on guidelines from the Third International Consensus (Sepsis-3) Definitions (4). The presence of AKI was determined according to Kidney Disease Improving Global Outcomes (KDIGO) criteria (46). We obtained Information on patients' pre-existing conditions, including diabetes, chronic lung disease, tumors, cardiovascular disease, hypertension, cerebrovascular disease, chronic liver disease, and chronic kidney disease. We excluded patients with a history of drug abuse, recurring infections, immunodeficiency, pregnancy, renal replacement therapy, discharge from ICU in previous year and organ transplantation. We calculated survival and AKI from the day of admission to the ICU until death or discharge up to 28 days. All participants or their proxies provided written informed consent, and patient data were de-identified and stored on a password-protected research computer.

### Biochemical Measurements

We collected blood samples for biochemical analysis from the 87 patients with septic shock within six hours of ICU admission, with baseline samples obtained on the day of patient admission to the ICU (day one) and additional samples obtained on days two and three. We collected only baseline blood samples from healthy volunteers. Blood samples were centrifuged at 1,000×g for 15 minutes at 2-8° C., and the plasma supernatant collected and stored at −80° C. We used enzyme-linked immunosorbent assay (ELISA) to measure serum Gal-3 (R&D Systems, Minneapolis, MN, USA) and serum IL-6 (Elabscience Biotech, Wuhan, Hubei, China) concentrations. Serum creatinine (Cr) was measured using a Cr detection kit (Cat. No. E-BC-K139-S, Elabscience, Wuhan, China). We collected urine samples from 54 (62%) randomly selected patients with sepsis to analyze urine Gal-3 concentrations.

### Sepsis-Related Parameters

To assess illness severity and predict patient prognosis, we determined Sequential Organ Failure Assessment (SOFA) score upon patient admission to the ICU (Seymour et al., 2016). Additionally, we calculated the Acute Physiology and Chronic Health Evaluation II (APACHE II) within 24 hours of admission to the ICU (47, 48). We measured point-of-care serum lactate concentrations at the bedside using the fingertip device (StatStrip® Lactate, Nova Biomedical Corporation, Waltham, MA, USA), reported as mmol/L.

### Animal Studies

### 1. Rat Cecal Ligation and Puncture (CLP) Model of Sepsis and Apheresis

A monoclonal antibody targeting rat Gal-3 demonstrated high specificity and affinity, as verified by both Biacore technology for detailed affinity and kinetic analysis, as well as Ex-vivo depletion of rat Gal-3. The proprietary antibody (Eliaz Therapeutics Inc., Santa Rosa California, 95401) was selected through a meticulous development process involving isolation and purification of various clones from rabbits immunized against Gal-3, with each clone showing promising initial binding characteristics. The best-performing monoclonal antibody was then expressed and produced using an established Chinese Hamster Ovary (CHO) cell line (49). The purified antibody was then attached to agarose beads and loaded into columns through which whole blood could flow using connections compatible with standard apheresis equipment. An empty column was used as a sham for controls.

### Sepsis Induction Using Cecal Ligation and Puncture

Standardized induction of sepsis in animal models can be carried out via cecal ligation and puncture or lipopolysaccharide (LPS) injection (34, 42, 50-52). Nineteen male adult Sprague-Dawley rats (400-600 gr) were obtained from the Animal Care and Use Committee of Wuhan University. We induced sepsis in all rats using cecal ligation and puncture (CLP) (52) by ligating 25% of the cecum followed by two punctures using a 20-gauge needle inferior to the ileocecal valve. After closure of the abdominal wall, rats were resuscitated with 20 mL/kg pre-warmed saline subcutaneously.

### Removal of Gal-3 Using Apheresis

The rat CLP model served as a preliminary study to demonstrate Gal-3 removal via selective withdrawal in a sepsis model using an adsorption column. The study utilized ten rats in the treatment group and nine rats in the control group. All rats (n=19) were anesthetized with isoflurane. Blood was drawn from the right femoral vein of rats and passed through the rat Gal-3 apheresis column (treatment group) or through an empty sham column (control group) then returned via the jugular vein. The blood rate was maintained at 0.8-1.0 mL/min by a mini-pump. Due to inherently low plasma volumes in rats, it was not viable to perform plasma separation. As a result, whole blood apheresis was used. It was started 60 minutes after CLP and lasted for two hours.

### Survival Measures

The survival rate was recorded daily in Gal-3 whole blood apheresis and sham groups during the 7 days following CLP. Measurements were recorded in timely and periodic repetitions as determined by the study size and complexity.

### 2. Porcine LPS Model of Sepsis and Apheresis

Preparation of plasma adsorption column for Gal-3 removal in pigs Apheresis columns prepared for selective withdrawal of gal-3 were prepared in a fashion and design not dissimilar from other columns discussed herein.

The XGAL-3 technology is intended to offer an apheresis adsorber for selectively reducing bound and unbound circulating Gal-3. Eliaz Therapeutics Inc. developed an ex vivo immunoadsorption column utilizing a human anti-Gal-3 antibody developed using a CHO expression system of the CHO-K1 cell line. The antibody was 100% pure by size exclusion-high-performance liquid chromatography (SEC-HPLC). The affinity of the antibody to porcine Gal-3 was confirmed in both Ex vivo and In vivo studies. The purified antibody was attached to agarose beads and loaded into columns (XGAL-3®, Eliaz Therapeutics Inc, Santa Rosa, California, 95401).

### Sepsis Induction Using Lipopolysaccharide

Lipopolysaccharides are endotoxins and outer cell components of gram-negative bacteria that activate an acute systemic inflammatory response by triggering the release of inflammatory cytokines and immune mediators (42, 50); these substances are commonly used to produce a sepsis-like physiology without infection (34), as demonstrated via intravenous infusion in a porcine model (41). Thirty one male Bama miniature pigs (Sus scrofa domestica; Hubei Yizhicheng Biotechnology Co., Ltd.) weighing approximately 30 kg underwent LPS-mediated induction of sepsis (41). On procedure day, the animals were intubated, anesthetized, and prepared surgically, including insertion of a central venous catheter, a Swan Ganz catheter, and both femoral artery and internal jugular vein catheters. There was a subsequent 60-minute period of stabilization and recovery period. Thereafter, we started an intravenous LPS infusion at 2 μg/Kg/h which was increased to 4 μg/Kg/h after 10 minutes and maintained over 20 additional minutes for a total of 30 minutes. Following this step, there was 30 minutes of recovery time. The animals then commenced the LPS acceleration phase, during which LPS infusion rate resumed at 6 μg/Kg/h for 10 minutes, followed by a dose increase to 12 μg/Kg/h for 10 minutes, and then 24 μg/Kg/h for an additional 25 minutes, for a total of 45 minutes. The LPS infusion was then discontinued, and the animals underwent fluid resuscitation and pressor support as indicated.

### Removal of Gal-3 Using Apheresis

A dual-lumen dialysis catheter was inserted into the femoral vein. Ten minutes after commencing the acceleration phase, the dialysis catheter was opened and plasma adsorption started using the Spectra Optia blood cell separation system, with plasma flow rate controlled at 25 ml/minute. Sixteen animals (treatment group) underwent selective Gal-3 depletion via therapeutic selective withdrawal apheresis using the XGAL-3 column loaded with monoclonal anti Gal-3 antibody (FIG. 1). Fifteen animals (control group) underwent sham apheresis via plasma separation without filtration through columns (FIG. 1). The apheresis treatment started 10 minutes after the start of the acceleration phase (with the LPS dose of 12 mcg/kg/h) and lasted for 3 h after the final dose of LPS, for a total of 3 h and 35 minutes. The fluid repletion of animals during resuscitation and supportive care consisted of 0.9% NaCl solution, dextran-40, and a 5% glucose solution. The fluid intake during the trial period was responsible for normalizing the blood pressure levels in the animals, together with the use of norepinephrine as a pressor. Throughout LPS induction and apheresis, blood samples were obtained every 60 minutes. To uphold the standard-of-care rationale, the pigs were provided with ventilation support, vasopressors (e.g., norepinephrine) (53) and fluid resuscitation for the first 6 hours. The animals were then transported to the animal facility and, once awake, extubated and hemodynamically stable, were provided with regular water and food for the remainder of the study. Heart rate (HR), blood pressure (BP), mean arterial pressure (MAP) and cardiac index (CI) were measured throughout the experiment. Mortality was recorded at 3, 4, 5, 6, 12 and 24 hours, after which all surviving pigs were euthanized, and tissue samples were taken from the kidney, heart, liver, and lung for comparative histological analysis.

### Sepsis-Related Measures

Extravascular Lung Water Index (ELWI) is an important indicator in systemic sepsis-associated inflammation and was used to quantify the amount of water in the lungs outside of the blood vessels (54, 55). Lactate, a key biomarker of sepsis severity (56), was measured using blood samples. Gal-3 and IL-6 concentrations were determined from blood samples. In addition, we assessed the survival rate over the 24 hrs following sepsis induction. Post termination samples of lung, heart, liver, and kidney tissue were obtained and examined histologically using H&E staining. Biopsy Interpretation was provided by a histopathologist blinded to the treatment groups.

### Statistics

We expressed normally distributed continuous variables as mean±SEM, and intergroup differences were analyzed using one-way analysis of variance (ANOVA) or independent sample t-test. We compared skewed continuous variables using the Mann-Whitney U test and expressed as median+interquartile range (IQR) following confirmation by Shapiro-Wilk test. P values of 0.05 or less were considered statistically significant.

We adjusted for potential confounding effects, including age, and pre-existing conditions, using Generalized Linear Models (GLMs) with a Gamma distribution. We used Binomial GLMs with a binary outcome for mortality and AKI to examine the relationship of Gal-3 with likelihood of mortality and AKI. Additionally, we examined the relations between Gal-3 and continuous variables using Spearman's rank correlation coefficient and used Mann-Whitney U tests to examine differences in the distributions of continuous variables.

We plotted survival rates of sham and treatment groups using the Kaplan-Meier product limit method and were compared using the log-rank test. We considered a 2-sided p<0.05 statistically significant. All statistical analyses were performed using R software version 4.2.3 (57).

These studies, as well as prior testing, focus on the selective withdrawal or selective removal of Gal-3. As shown, this is affected through a primary apheresis column. Multiple columns may be used, as disclosed in prior patents including U.S. Pat. No. 10,953,148. These secondary columns offer the opportunity to “fine tune” treatments as well as advance and support treatment through selective withdrawal (the terms selective withdrawal and selective removal are used interchangeably herein to refer to the process employed, where apheresis is used to reduce levels of Gal-3 or other targets). Thus, in addition to selective withdrawal/removal of Gal-3, the invention further comprises the selective withdrawal of other targets, including inflammatory compounds, cytokines and LPS via whole blood or plasma apheresis. The invention specifically contemplates, in addition to removal of Gal-3, the selective withdrawal of LPS, CRP, different cytokines, and microbial agents (whole blood) including withdrawal of specific targets including lipopolysaccharides (LPS) C-Reactive Protein, IL-4, IL-6, IL-1B, IL-10, TNF alpha, NF-kB, High Mobility Group Protein B1 (HMGB1) and combinations thereof. More generally, other cytokines may also be addressed by selective withdrawal, including various chemokines, interferons, other interleukins and the like. These include without limitation Interleukin-1 (IL-1), Interleukin-10 (IL-10), Interleukin-8 (IL-8), Interleukin-12 (IL-12), Interferon-gamma (IFN-γ), Monocyte Chemoattractant Protein-1 (MCP-1/CCL2), Transforming Growth Factor-beta (TGF-β) and Interleukin-17 (IL-17). These and similar agents are discussed in www.sinobiological.com/resource/cytokines/role-of-cytokines-in-sepsis.

As also described in patents similar to U.S. Pat. No. 10,953,148 and many other texts, effective treatment may depend not only on selective removal of gal-3 from the blood, and other blood agents, but it may depend on the administration or addition of therapeutic or beneficial elements before the blood is returned to the mammal. Often, addition of the agent to the plasma is an effective and efficient means of administration of the treatment, such as a medication, vitamin, or similar component. Thus, while not essential to the selective removal of gal-3, the methods set forth in this disclosure include o administration of a variety of agents to the patient, which may be effectively achieved by addition to plasma and/or whole blood during the practice of apheresis. Examples include various homeopathic medications, metabolism regulators, immune reaction modifiers, pharmaceuticals and various cytotoxic compounds, anti-microbial agents, chelating agents, possibly targeted ones, to aid in treatment.

Acute kidney injury (AKI) characterizes a situation when a mammal's kidneys suddenly can't filter waste products from the blood. When the kidneys can't filter wastes, harmful levels of wastes may build up. The blood's chemical makeup may get out of balance. Acute kidney injury used to be called acute kidney failure. Acute kidney injury is most common in people who are in the hospital, mostly in people who need intensive care. Acute kidney injury ranges from mild to severe. If severe, ongoing and not treated, it can be fatal. As disclosed herein, however, AKI it also can be reversed. People in otherwise good health may get back typical or nearly typical use of their kidneys.

The studies detailed herein demonstrate the effectiveness of the treatment of disease conditions previously difficult to treat or beyond treatment. Apheresis to achieve selective withdrawal of Gal-3 is shown herein to be an effective treatment of sepsis. Given that sepsis is often encountered in a hospital or clinic setting, the ability to provide the septic patient with apheresis complements the effective remedy set forth herein. In addition, our research supports the effective treatment of acute kidney injury (AKI) associated sepsis. AKI is frequently encountered in a septic patient, complicating treatment and effective recovery. By treating the patient with selective withdrawal of Gal-3 to reduce or at least control serum levels of Gal-3, both sepsis and sepsis induced AKI related illness may be treated. Beyond sepsis based illness and treatment, this application demonstrates the effective treatment of AKI per se, independent of sepsis related complications. Treatment of AKI, with or without sepsis related issues and complications, present additional opportunities for effective treatment using selective withdrawal of Gal-3 achieved through apheresis.

### REFERENCES

To provide readers and researchers the opportunity to review and develop additional information, all references identified in this application are recited below. These references are incorporated herein by reference, to provide for complete understanding and review.
1. Kellum J A, Ronco C. The role of endotoxin in septic shock. Crit Care. 2023; 27(1):400.
2. Martin G S, et al. The Epidemiology of Sepsis in the United States from 1979 through 2000. N Engl J Med. 2003; 348(16):1546-1554.
3. Prescott H C, Angus D C. Enhancing Recovery From Sepsis: A Review. JAMA. 2018; 319(1):62.
4. Singer M, et al. The Third International Consensus Definitions for Sepsis and Septic Shock (Sepsis-3). JAMA. 2016; 315(8):801.
5. Schlapbach L J, et al. World Sepsis Day: a global agenda to target a leading cause of morbidity and mortality. American Journal of Physiology-Lung Cellular and Molecular Physiology. 2020; 319(3):L518-L522.
6. Rudd K E, et al. Global, regional, and national sepsis incidence and mortality, 1990-2017: analysis for the Global Burden of Disease Study. The Lancet. 2020; 395(10219):200-211.
7. Bellomo R, et al. Acute kidney injury in sepsis. Intensive Care Med. 2017; 43(6):816-828.
8. Lafrance J-P, Miller D R. Acute Kidney Injury Associates with Increased Long-Term Mortality. Journal of the American Society of Nephrology. 2010; 21(2):345-352.
9. Uchino S. Acute Renal Failure in Critically Ill PatientsA Multinational, Multicenter Study. JAMA. 2005; 294(7):813.
10. Wang H E, et al. Acute Kidney Injury and Mortality in Hospitalized Patients. Am J Nephrol. 2012; 35(4):349-355.
11. Hoste E A J, et al. Epidemiology of acute kidney injury in critically ill patients: the multinational AKI-EPI study. Intensive Care Med. 2015; 41(8):1411-1423.
12. Polat G, et al. Sepsis and Septic Shock: Current Treatment Strategies and New Approaches. Eurasian J Med. 2017; 49(1):53-58.
13. Evans L, et al. Surviving sepsis campaign: international guidelines for management of sepsis and septic shock 2021. Intensive Care Med. 2021; 47(11):1181-1247.
14. Rivers E P. Early and innovative interventions for severe sepsis and septic shock: taking advantage of a window of opportunity. Canadian Medical Association Journal. 2005; 173(9):1054-1065.
15. Delano M J, Ward P A. Sepsis-induced immune dysfunction: can immune therapies reduce mortality? Journal of Clinical Investigation. 2016; 126(1):23-31.
16. Kashiouris M G, et al. The Emerging Role of Vitamin C as a Treatment for Sepsis. Nutrients. 2020; 12(2):292.
17. McAuley D, Charles P-E, Papazian L. Statins in patients with sepsis and ARDS: is it over?We are not sure. Intensive Care Med. 2017; 43(5):677-679.
18. Ranieri V M, et al. Drotrecogin Alfa (Activated) in Adults with Septic Shock. N Engl J Med. 2012; 366(22):2055-2064.
19. Kharga K, Kumar L, Patel S K S. Recent Advances in Monoclonal Antibody-Based Approaches in the Management of Bacterial Sepsis. Biomedicines. 2023; 11(3):765.
20. Henderson N C, Sethi T. The regulation of inflammation by galectin-3. Immunological Reviews. 2009; 230(1):160-171.
21. Wang Z, et al. Melatonin inhibits atherosclerosis progression via galectin-3 downregulation to enhance autophagy and inhibit inflammation. Journal of Pineal Research. 2023; 74(3):e12855.
22. Zhang Y, et al. Locally delivered modified citrus pectin—a galectin-3 inhibitor shows expected anti-inflammatory and unexpected regeneration-promoting effects on repair of articular cartilage defect. Biomaterials. 2022; 291:121870.
23. Humphries D C, et al. Galectin-3 inhibitor GB0139 protects against acute lung injury by inhibiting neutrophil recruitment and activation. Front Pharmacol. 2022; 13:949264.
24. Liu Y, et al. Galectin-3 regulates microglial activation and promotes inflammation through TLR4/MyD88/NF-kB in experimental autoimmune uveitis. Clinical Immunology. 2022; 236:108939.
25. Simovic Markovic B, et al. Galectin-3 Plays an Important Pro-inflammatory Role in the Induction Phase of Acute Colitis by Promoting Activation of NLRP3 Inflammasome and Production of IL-1β in Macrophages. ECCOJC. 2016; 10(5):593-606.
26. Siwicki M, Engblom C, Pittet M J. Gal3 Links Inflammation and Insulin Resistance. Cell Metabolism. 2016; 24(5):655-656.
27. Ferreira R G, et al. Galectin-3 aggravates experimental polymicrobial sepsis by impairing neutrophil recruitment to the infectious focus. Journal of Infection. 2018; 77(5):391-397.
28. Mishra B B, et al. Galectin-3 Functions as an Alarmin: Pathogenic Role for Sepsis Development in Murine Respiratory Tularemia. PLoS ONE. 2013; 8(3):e59616.
29. Wu M-H, et al. Galectin-1 induces vascular permeability through the neuropilin-1/vascular endothelial growth factor receptor-1 complex. Angiogenesis. 2014; 17(4):839-849.
30. Karabacak P. Serum galectin-3 levels predict poor prognosis in sepsis and septic shock patients. Rev Assoc Med Bras. 2023; 69(8):e20220940.
31. Wang F, et al. Galectin-3 Mediates Endotoxin Internalization and Caspase-4/11 Activation in Tubular Epithelials and Macrophages During Sepsis and Sepsis-Associated Acute Kidney Injury. Inflammation. 2024; 47(1):454-468.
32. Volarevic V, et al. Galectin 3 protects from cisplatin-induced acute kidney injury by promoting TLR-2-dependent activation of IDO1/Kynurenine pathway in renal DCs. Theranostics. 2019; 9(20):5976-6001.
33. Sun H, et al. Galectin-3 in septic acute kidney injury: a translational study. Crit Care. 2021; 25(1):109.
34. Fernández-Martín J C, et al. Gal3 Plays a Deleterious Role in a Mouse Model of Endotoxemia. IJMS. 2022; 23(3):1170.
35. Navarro-Alvarez N, et al. The effects of galectin-3 depletion apheresis on induced skin inflammation in a porcine model. J of Clinical Apheresis. 2018; 33(4):486-493.
36. Sun Z, et al. The dynamic changes of gal-3 predict the outcome of septic acute kidney injury. Critical Care Medicine. 2024; 52(1):S689-S689.
37. Shiga H, et al. Continuous Hemodiafiltration with a Cytokine-Adsorbing Hemofilter in Patients with Septic Shock: A Preliminary Report. Blood Purif. 2014; 38(3-4):211-218.
38. Poole D, D'Arrigo S. Coupled plasma filtration and adsorption for the treatment of septic shock. Intensive Care Med. 2022; 48(4):493-495.
39. Ronco C, et al. Adsorption in sepsis. Kidney International. 2000; 58:S148-S155.
40. Schittek G A, et al. Adsorption therapy in critically ill with septic shock and acute kidney injury: a retrospective and prospective cohort study. Ann Intensive Care. 2020; 10(1):154.
41. Gomez H, et al. The Microcirculatory Response to Endotoxemia and Resuscitation Is a Marker of Regional Renal Perfusion, Renal Metabolic Stress, and Tubular Injury. Antioxidants & Redox Signaling. 2021; 35(17):1407-1425.
42. Wyns H, et al. In vivo porcine lipopolysaccharide inflammation models to study immunomodulation of drugs. Veterinary Immunology and Immunopathology. 2015; 166(3-4):58-69.
43. Lerolle N, et al. Histopathology of septic shock induced acute kidney injury: apoptosis and leukocytic infiltration. Intensive Care Med. 2010; 36(3):471-478.
44. Martinez-Martinez E, et al. Galectin-3 Blockade Reduces Renal Fibrosis in Two Normotensive Experimental Models of Renal Damage. PLoS ONE. 2016; 11(11):e0166272.
45. Parikh C R, et al. Postoperative Biomarkers Predict Acute Kidney Injury and Poor Outcomes after Adult Cardiac Surgery. Journal of the American Society of Nephrology. 2011; 22(9):1748-1757.
46. Kellum J A, et al. Kidney disease: Improving global outcomes (KDIGO) acute kidney injury work group. KDIGO clinical practice guideline for acute kidney injury. Kidney International Supplements. 2012; 2(1):1.
47. Gall J-R L, Loirat P, Alperovitch A. APACHE II-A Severity of Disease Classification System: Critical Care Medicine. 1986; 14(8):754.
48. Rivers E, et al. Early Goal-Directed Therapy in the Treatment of Severe Sepsis and Septic Shock. N Engl J Med. 2001; 345(19):1368-1377.
49. Bailey L A, et al. Determination of Chinese hamster ovary cell line stability and recombinant antibody expression during long-term culture. Biotech & Bioengineering. 2012; 109(8):2093-2103.
50. Dickson K, Lehmann C. Inflammatory Response to Different Toxins in Experimental Sepsis Models. IJMS. 2019; 20(18):4341.
51. Otero-Antón E, et al. Cecal Ligation and Puncture as a Model of Sepsis in the Rat: Influence of the Puncture Size on Mortality, Bacteremia, Endotoxemia and Tumor Necrosis Factor Alpha Levels. Eur Surg Res. 2001; 33(2):77-79.
52. Toscano M G, Ganea D, Gamero A M. Cecal Ligation Puncture Procedure. JoVE. 2011; (51):2860.
53. Russell J A. Vasopressor therapy in critically ill patients with shock. Intensive Care Med. 2019; 45(11):1503-1517.
54. Jozwiak M, Teboul J-L, Monnet X. Extravascular lung water in critical care: recent advances and clinical applications. Ann Intensive Care. 2015; 5(1):38.
55. Luo P, et al. Evaluation of the association between extravascular lung water and prognosis of sepsis: A protocol of systematic review. Medicine. 2021; 100(5):e23971.
56. Faix J D. Biomarkers of sepsis. Critical Reviews in Clinical Laboratory Sciences. 2013; 50(1):23-36.
57. R Core Team. R: A Language and Environment for Statistical Computing. R Foundation for Statistical Computing. Vienna, Austria.
58. Wickham H, et al. dplyr: A Grammar of Data Manipulation. R package version 1.1.4, [Internet]. 2023. <https://CRAN.R-project.org/package=dplyr>.
59. Wickham H. ggplot2: Elegant Graphics for Data Analysis. New York, NY: Spinger-Verlag; 2016.
60. Xu S, et al. Use ggbreak to Effectively Utilize Plotting Space to Deal With Large Datasets and Outliers. Front Genet. 2021; 12:774846.
61. Kassambara A. ggpubr: “ggplot2” Based Publication Ready Plots. R package version 0.6.0, [Internet]. 2023. <https://CRAN.R-project.org/package=ggpubr.
62. Kassambara A. rstatix: Pipe-Friendly Framework for Basic Statistical Tests. R package version 0.7.2, [Internet]. 2023. <https://CRAN.R-project.org/package=rstatix>.
63. Lüdecke D. ggeffects: Tidy Data Frames of Marginal Effects from Regression Models. JOSS. 2018; 3(26):772.

The various embodiments have been described in detail with reference to the accompanying drawings. FIGS. 1-10 submitted herewith provide detailed information regarding the three trials elide upon and described herein. Wherever possible, we have made an effort to avoid repetition or overlap between one set of results and another.

While the present invention has been disclosed both generically, and with reference to specific alternatives, those alternatives are not intended to be limiting unless reflected in the claims set forth below. The invention is limited only by the provisions of the claims, and their equivalents, as would be recognized by one of skill in the art to which this application is directed.

What is claimed is:

1. A method of treating sepsis in a mammal, comprising:
a) Identifying a mammalian patient in need of treatment for sepsis,
b) Administering apheresis comprising selective withdrawal of galectin-3 (Gal-3) to said mammalian patient so identified repeatedly for a period of time sufficient to reduce the Gal-3 level in the blood of said mammalian patient.

2. The method of claim 1, wherein said period of time is at least three days, in which said patient receives said apheresis treatment at least once in each day.

3. The method of claim 2, wherein said period of time is at least five days.

4. The method of claim 1, wherein said mammalian patient exhibits acute kidney injury (AKI) associated with said sepsis.

5. A method of treating acute kidney injury (AKI) in a mammal in need of same, comprising:
a) Identifying a mammalian patient in need of treatment for AKI,
b) Administering apheresis comprising selective withdrawal of Gal-3 to said mammalian patient so identified repeatedly for a period of time sufficient to reduce the Gal-3 level in the blood of said mammalian patient.

6. The method of claim 1, wherein treatment of said patient further comprises administration of at least one agent to said patient, wherein said agent is selected from the group consisting of metabolism regulators, immune reaction modifiers, pharmaceuticals, cytotoxic compounds, anti-microbial agents, chelating agents and combinations thereof.

7. The method of claim 5, wherein treatment of said patient further comprises administration of an agent to said patient, wherein said agent is selected from the group consisting of metabolism regulators, immune reaction modifiers, pharmaceuticals, cytotoxic compounds, anti-microbial agents, chelating agents and combinations thereof.

8. The method of claim 5, wherein treatment of said patient further comprises selective removal of at least one additional blood component selected from the group consisting of lipopolysaccharides (LPS), C-Reactive Protein, IL-4, IL-6, IL-1B, IL-10, TNF alpha, NF-kB, and combinations thereof.
